(19) United States
(12) Patent Application Publication
(10) Pub. No.: US 2011/0124032 A1
(43) Pub. Date: May 26, 2011

(54) Methods and Compositions for Treating Carcinoma Stem Cells

(76) Inventors: Maximilian Diehn, Stanford, CA (US); Michael Clarke, Palo Alto, CA (US); Irving L. Weissman, Stanford, CA (US)

(21) Appl. No.: 12/865,092

(22) PCT Filed: Jan. 30, 2009
(86) PCT No.: PCT/US09/00592

§ 371 Date: Feb. 8, 2011

Related U.S. Application Data
(60) Provisional application No. 61/063,384, filed on Feb. 1, 2008.

(51) Int. Cl. C12Q 1/02

(52) U.S. Cl. 435/29

(57) ABSTRACT

Cancer stem cells (CSCs) have been prospectively isolated or identified from primary tumor samples, and shown to possess the unique properties of self-renewal and differentiation, and can form unique histological microdomains useful in cancer diagnosis. Such cancer stem cells are shown herein to have the phenotype of containing decreased levels of reactive oxygen species (ROS) relative to non-tumorigenic (non-stem cell) cancer cells, as well as expression of other protective pathways. The CSCs are further shown to be more resistant to ionizing radiation (IR) and certain chemotherapies and to express high levels of ROS genes.

### GOVERNMENT RIGHTS

This invention was made with Government support under contract CA126524 awarded by the National Cancer Institute. The Government has certain rights in this invention.

### BACKGROUND OF THE INVENTION

Basic cancer research has focused on identifying the genetic changes that lead to cancer. This has led to major advances in our understanding of the molecular and biochemical pathways that are involved in tumorigenesis and malignant transformation. However, our understanding of the cellular biology has lagged. Although the effects of particular mutations on the proliferation and survival of model cells, such as fibroblasts or cell lines, can be predicted, the effects of such mutations on the actual cells involved in specific cancers is largely guesswork.

A tumor can be viewed as an aberrant organ initiated by a tumorigenic cancer cell that acquired the capacity for indefinite proliferation through accumulated mutations. In this view of a tumor as an abnormal organ, the principles of normal stem cell biology can be applied to better understand how tumors develop. Many observations suggest that analogies between normal stem cells and tumorigenic cells are appropriate. Both normal stem cells and tumorigenic cells have extensive proliferative potential and the ability to give rise to new (normal or abnormal) tissues. Both tumors and normal tissues are composed of heterogeneous combinations of cells, with different phenotypic characteristics and different proliferative potentials.

Because most tumors have a clonal origin, the original tumorigenic cancer cell gives rise to phenotypically diverse progeny, including cancer cells with indefinite proliferative potential, as well as cancer cells with limited or no proliferative potential. This suggests that tumorigenic cancer cells undergo processes that are analogous to the self-renewal and differentiation of normal stem cells. Tumorigenic cells can be thought of as cancer stem cells that undergo an aberrant and poorly regulated process of organogenesis analogous to what normal stem cells do. Although some of the heterogeneity in tumors arises as a result of continuing mutagenesis, it is likely that heterogeneity also arises through the aberrant differentiation of cancer cells.

It is well documented that many types of tumors contain cancer cells with heterogeneous phenotypes, reflecting aspects of the differentiation that normally occurs in the tissues from which the tumors arise. The variable expression of normal differentiation markers by cancer cells in a tumor suggests that some of the heterogeneity in tumors arises as a result of the anomalous differentiation of tumor cells. Examples of this include the variable expression of myeloid markers in chronic myeloid leukaemia, the variable expression of neuronal markers within peripheral neurectodermal tumors, and the variable expression of milk proteins or the oestrogen receptor within breast cancer.

Tumorigenic and non-tumorigenic populations of breast cancer cells can be isolated based on their expression of cell surface markers. In many cases of breast cancer, only a small subpopulation of cells had the ability to form new tumors. This work strongly supports the existence of cancer stem cells (CSC) in breast cancer. Further evidence for the existence of CSC occurring in solid tumors has been found in central nervous system (CNS) malignancies. Using culture techniques similar to those used to culture normal neuronal stem cells it has been shown that neuronal CNS malignancies contain a small population of cancer cells that are clonogenic in vitro and initiate tumors in vivo, while the remaining cells in the tumor do not have these properties.

Stem cells are defined as cells that have the ability to perpetuate themselves through self-renewal and to generate mature cells of a particular tissue through differentiation. In most tissues, stem cells are rare. As a result, stem cells must be identified prospectively and purified carefully in order to study their properties. Perhaps the most important and useful property of stem cells is that of self-renewal. Through this property, striking parallels can be found between stem cells and cancer cells: tumors may often originate from the transformation of normal stem cells, similar signaling pathways may regulate self-renewal in stem cells and cancer cells, and cancers may comprise rare cells with indefinite potential for self-renewal that drive tumorigenesis.

The presence of CSC has profound implications for cancer therapy. A small number of disseminated cancer cells can be detected at sites distant from primary tumors in patients that never manifest metastatic disease. One possibility is that most cancer cells lack the ability to form a new tumor such, that only the dissemination of rare CSC can lead to metastatic disease.

Currently available therapeutics, including radiotherapy and chemotherapy, were generally developed and optimized by considering all cells within a tumor to have unlimited proliferative potential and similar sensitivities. However, CSCs may be intrinsically more resistant to ionization radiation (IR) and other cytotoxic agents than non-CSCs, based on the notion that CSCs are likely share properties of normal tissue stem cells, which must have an innate resistance to apoptosis, toxins, and DNA damage in order to be able to survive for the life of an organism. In light of this principle, the goal of therapy must be to identify and kill this cancer stem cell population.

The prospective identification and isolation of cells resistant to chemotherapy or IR will allow more targeted and effective cancer therapy. Existing therapies have been developed largely against the bulk population of tumor cells, because the therapies are identified by their ability to shrink the tumor mass. However, because most cells within a cancer have limited proliferative potential, an ability to shrink a tumor mainly reflects an ability to kill these cells. Therapies that are more specifically directed against CSC will result in more durable responses and cures of metastatic tumors. It is therefore essential that we develop a deeper understanding of the biology of this disease in order to develop more effective therapies.

References of interest include, without limitation, a discussion of BM1 by Lessard, & Sauvageau (2003) Nature 423, 255-60; Molofsky et al. (2003) Nature 425, 962-7; Park et al. (2004) J Clin Invest 113, 175-9; Park et al. (2003) Nature 423, 302-5; and Valk-Lingbeek, M. E., Bruggeman, S. W. & van Lohuizen, M. (2004) Cell 118, 409-18.

Cancer Stem cells are discussed in, for example, Pardal et al. (2003) Nat Rev Cancer 3, 895-902; Reya et al. (2001) Nature 414, 105-11; Bonnet & Dick (1997) Nat Med 3, 730-7; Al-Hajj et al. (2003) Proc Natl Acad Sci USA 100, 3983-8; Dontu et al. (2004) Breast Cancer Res 6, R605-15; Singh et al. (2004) Nature 432, 396-401.

### SUMMARY OF THE INVENTION

Cancer stem cells (CSCs) have been prospectively isolated or identified from primary tumor samples, and shown to possess the unique properties of self-renewal and differentiation, and can form unique histological microdomains useful in cancer diagnosis. Such cancer stem cells are shown herein to have the phenotype of containing decreased levels of reactive oxygen species (ROS) relative to non-tumorigenic (non-stem cell) cancer cells, and to express high levels of ROS genes, as well as expression of other protective pathways. The CSCs are further shown to be more resistant to ionizing radiation (IR) and certain chemotherapies, herein collectively referred to as cytotherapy.

In some embodiments, a tumor sample is analyzed, e.g. by histochemistry, including immunohistochemistry, in situ hybridization, and the like, for the presence of CSC that have decreased levels of ROS or increased expression of protective pathway genes such as ROS genes, which include, without limitation, cytochrome b alpha subunit (CYBA); prion protein (PRNP); peptide methionine sulfoxide reductase (MSRA); glutathione peroxidase 1 (GPX1); thioredoxin-interacting protein (TXNIP); superoxide dismutase 2 (SOD2); catalase (CAT); xpa gene (XPA); isocitrate dehydrogenase 1 (IDH1); and glutathione peroxidase 4 (GPX4). In some embodiment the tumor sample is also stained with one or more markers that characterize cancer stem cells. The staining may be performed on a biopsy section, isolated cells, etc. Optionally the CSC present in the sample are separated from non-tumorigenic cells; and the presence of ROS or ROS genes is determined in the stem cell fraction. An increased number of CSC having decreased levels of ROS relative to non-tumorigenic cancer cells is indicative that the tumor is more resistant to ionizing radiation and certain chemotherapies. The phenotypic features of CSCs described herein provide a means of predicting disease progression, relapse, and development of drug resistance.

In some embodiments of the invention, therapeutic compositions for cancer are provided. The ROS genes that are found to be overrepresented in CSCs can be effective targets to treat invasive cancer. High expression level of these ROS genes allow high tolerance of IR and chemotherapy by CSCs. Decreased expression of these ROS genes disclosed within can lead to more effective cell killing of CSCs exposed to IR or chemotherapy.

In another embodiment of the invention, methods are provided to screen compounds that decrease CSCs' tolerance of IR and chemotherapy. CSCs may be used, for example, in a method of screening a compound that increases the level of ROS above that of non-tumorigenic cancer cells. This involves combining the compound with the CSCs disclosed herein, and then determining any modulatory effect resulting from the compound. This may include examination of the cells for viability, toxicity, metabolic change, or an effect on cell function in response to chemotherapy drugs or IR.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-F. Analysis of ROS levels in normal mammary and breast cancer stem cells and their progeny. a, CD24^{med}CD49f^{high}Lin⁻ mammary cells (mammary stem cell-containing population) and CD24^{high}CD49f^{low}Lin⁻ mammary cells (progenitor cell-containing population) were isolated from C57BI/6J female mice using flow cytometry and intracellular ROS concentrations were measured by DCF-DA staining. b, as in a but using 29S1/SvImJ mice. c, Mean±s.e.m. for replicates of a and b (n=6; p=0.001). d, as in b but using MitoSOX Red instead of DCF-DA. Data shown are representative of two independent experiments. e, CD44⁺CD24^{-/low}Lin⁻ breast cancer cells (CSC-containing population) and “Not CD44⁺CD24^{-/low}” Lin⁻ cells (non-tumorigenic population) were isolated from a primary human breast tumor by flow cytometry and ROS levels were analyzed using DCF-DA. f, as in e but using murine Thy1⁺CD24⁺Lin⁻ breast cancer cells (CSC-containing population) and “Not Thy1⁺CD24⁺” Lin⁻ cells (non-tumorigenic population) isolated from an MMTV-Wnt-1 breast tumor.

FIG. 2. Thy1⁺CD24⁺Lin⁻ CSC-enriched cells develop less DNA damage after irradiation than non-tumorigenic cells. a, Thy1⁺CD24⁺Lin⁻ cells (CSC enriched population) and “Not Thy1⁺CD24⁺” Lin⁻ non-tumorigenic cells were isolated from MMTV-Wnt-1 breast tumors by flow cytometry and irradiated with 10 Gy of IR. DNA damage was measured before irradiation, immediately after irradiation, and 16 hrs later using the alkaline comet assay. Mean of median tail moments±s.e.m. (n=3; p=0.05). b, Using the data from a, the difference in median tail moments between the untreated and “no repair” time points was calculated. Mean±s.e.m. (n=3; p=0.004). c, Thy1⁺CD24⁺Lin⁻ cells and “Not Thy1⁺CD24⁺” Lin⁻ non-tumorigenic cells from MMTV-Wnt-1 tumors that were harvested 15 minutes after being irradiated in vivo with 1 Gy of IR were immunostained for γ-H2AX, a marker of DNA double strand breaks. Mean±s.e.m. (n=2; p=0.04).

FIG. 3. Enrichment of CSCs after in vivo irradiation. a, Breast tumors from MMTV-Wnt-1 mice were irradiated in vivo with 3×5 Gy or 5×2 Gy. Percentage of Thy1⁺CD24⁺Lin⁻ cells in untreated and irradiated tumors were quantified by flow cytometry 36 hours after the last fraction was delivered. b, Mean±s.e.m. for replicates of a (n=6; p=0.008). c, First generation xenografts established from two different primary human head and neck cancers were irradiated in vivo with 2×3 Gy. Percentage of CD44⁺Lin⁻ cells was quantified as above.

FIG. 4. Thy1⁺CD24⁺Lin⁻ cells over-express genes involved in ROS scavenging and pharmacologic modulation of ROS levels affects the radiosensitivity of Thy1⁺CD24⁺Lin⁻ and “Not Thy1⁺CD24⁺” Lin⁻ cells. a, Single cell qRT-PCR analysis of gene expression in Thy1⁺CD24⁺Lin⁻ CSC enriched cells and “Not Thy1⁺CD24⁺” Lin⁻ non-tumorigenic cells. The heatmap displays mean centered CT values. b, Clonogenic survival of Thy1⁺CD24⁺Lin⁻ CSC-enriched cells and “Not Thy1⁺CD24⁺” Lin⁻ non-tumorigenic cells before and after 2 Gy of ionizing radiation. Mean±s.e.m. (n=3; p=0.001). c, Clonogenic survival of “Not Thy1⁺CD24⁺” Lin⁻ non-tumorigenic cells in the presence or absence of the ROS scavenger tempol (10 mM). Mean±s.e.m. (n=2; p=0.03). d, Clonogenic survival of Thy1⁺CD24⁺Lin⁻ CSC-enriched cells in the presence or absence of 24 hour pre-treatment with the glutathione synthesis inhibitor L-S,R Buthionine Sulfoximine (BSO, 1 mM). Mean±s.e.m. (n=3; p=0.002). e, Clonogenic survival of Thy1⁺CD24⁺Lin⁻ CSC-enriched cells after 3 Gy of ionizing radiation with or without BSO pretreatment. Mean±s.e.m. (n=3; p=0.03).

### DETAILED DESCRIPTION OF THE EMBODIMENTS

Cancer stem cells from carcinomas are shown herein to have decreased levels of reactive oxygen species, (ROS) relative to non-tumorigenic cancer cells, as a result of high levels of expression of ROS related genes, e.g. cytochrome b alpha subunit (CYBA); prion protein (PRNP); peptide methionine sulfoxide reductase (MSRA); glutathione peroxidase (GPX1); thioredoxin-interacting protein (TXNIP); superoxide dismutase 2 (SOD2); catalase (CAT); xpa gene (XPA); isocitrate dehydrogenase 1 (IDH1); and glutathione peroxidase 4 (GPX4). The high expression level of these ROS genes and other protective pathways allows the cells to achieve a high tolerance of IR and chemotherapy.

In some embodiments the phenotypic features of CSCs described herein provide a means of predicting disease progression, relapse, and development of drug resistance. A tumor sample is analyzed, e.g. by flow cytometry, histochemistry, including immunohistochemistry, in situ hybridization, and the like, for the presence of CSC that have decreased levels of ROS or increased expression of ROS genes or other protective pathway genes. In some embodiments the tumor sample is stained with one or more markers that characterize cancer stem cells. Optionally the CSC present in the sample are separated from non-tumorigenic cells; and the presence of ROS or ROS genes is determined in the stem cell fraction.

In some embodiments of the invention, therapeutic compositions for cancer are provided. The ROS genes that are found to be overrepresented in CSCs can be effective targets to treat invasive cancer. Decreased expression of these ROS genes disclosed within can lead to more effective cell killing of CSCs exposed to IR or chemotherapy.

In another embodiment of the invention, methods are provided to screen compounds that decrease CSC tolerance of IR and chemotherapy. CSCs may be used, for example, in a method of screening a compound that increases the level of ROS above that of non-tumorigenic cancer cells. This involves combining the compound with the CSCs disclosed herein, and then determining any modulatory effect resulting from the compound. This may include examination of the cells for viability, toxicity, metabolic change, or an effect on cell function in response to chemotherapy drugs or IR.

The present invention identifies markers of cancer stem cells (CSC), including carcinoma stem cells. In one embodiment, the marker is a decreased level of intracellular ROS. In another embodiment the marker is polynucleotides, as well as polypeptides encoded thereby, that are differentially expressed in CSC. Polynucleotides of interest include ROS-related genes as described herein, and other protective pathway genes, including aldehyde dehydrogenase (ALDH), for example ALDH1A3; ABCB1 (P-glyoprotein/MDR1); BCL2A1; SNAI2 (slug); ATM, CHEK1, and CHEK2. Markers of particular interest are ROS related genes

Methods are provided in which these ROS, polynucleotides and polypeptides, which may be collectively referred to as CSC markers, are used for detecting, assessing, and reducing the growth of cancer cells. Methods may use one or a combination of markers, where a combination may include 2, 3 or more markers.

In some embodiments, the genetic and polypeptide markers are differentially expressed as a level at least 2× the expression level of a counterpart non-tumorigenic cell, where expression may be determined as the level of transcription, mRNA accumulation, and/or protein accumulation. In other embodiments the markers are expressed as a level at least 3×, at least 4×, at least 5×, at least 10×, at least 20× or greater, than the expression level of a counterpart non-tumorigenic cell. Where the marker is ROS, the level is reduced relative to a comparable non-tumorigenic cell, and may be reduced at least 2×, at least 3×, at least 4×, at least 10× or more.

The present invention provides methods of using the markers described herein in diagnosis of cancer, classification and treatment of cancers, particularly carcinomas. The methods are useful for characterizing CSC, facilitating diagnosis and the severity of the cancer (e.g., tumor grade, tumor burden, and the like) in a subject, facilitating a determination of the prognosis of a subject, and assessing the responsiveness of the subject to therapy. The detection methods of the invention can be conducted in vitro or in vivo, on isolated cells, or in whole tissues or a bodily fluid, e.g., blood, lymph node biopsy samples, and the like.

As used herein, the terms “a gene that is differentially expressed in a cancer stem cell,” and “a polynucleotide that is differentially expressed in a cancer stem cell”, are used interchangeably herein, and generally refer to a polynucleotide that represents or corresponds to a gene that is differentially expressed in a cancer stem cell when compared with a cell of the same cell type that is not cancerous, e.g., mRNA is found at levels at least about 25%, at least about 50% to about 75%, at least about 90%, at least about 1.5-fold, at least about 2-fold, at least about 3-fold, at least about 5-fold, at least about 10-fold, or at least about 50-fold or more, different (e.g., higher or lower). The comparison can be made between CSC and the non-tumorigenic counterpart cells. The term “a polypeptide marker for a cancer stem cell” refers to a polypeptide encoded by a polynucleotide that is differentially expressed in a cancer stem cell.

A polynucleotide or sequence that corresponds to, or represents a gene means that at least a portion of a sequence of the polynucleotide is present in the gene or in the nucleic acid gene product (e.g., mRNA or cDNA). A subject nucleic acid may also be “identified” by a polynucleotide if the polynucleotide corresponds to or represents the gene. Genes identified by a polynucleotide may have all or a portion of the identifying sequence wholly present within an exon of a genomic sequence of the gene, or different portions of the sequence of the polynucleotide may be present in different exons (e.g., such that the contiguous polynucleotide sequence is present in an mRNA, either pre- or post-splicing, that is an expression product of the gene). An “identifying sequence” is a minimal fragment of a sequence of contiguous nucleotides that uniquely identifies or defines a polynucleotide sequence or its complement.

The polynucleotide may represent or correspond to a gene that is modified in a cancer stem cell (CSC) relative to a normal cell. The gene in the CSC may contain a deletion, insertion, substitution, or translocation relative to the polynucleotide and may have altered regulatory sequences, or may encode a splice variant gene product, for example. The gene in the CSC may be modified by insertion of an endogenous retrovirus, a transposable element, or other naturally occurring or non-naturally occurring nucleic acid.

Sequences of interest include those set forth in Table 1, which are differentially expressed in CSC relative to normal counterpart cells:

| | Genbank | |
|---|---|---|
| Gene | reference | Description |
| cytochrome b alpha | NM_000101 | The CYBA gene encodes the alpha subunit, also known |
| subunit (CYBA) | | as the light chain, of cytochrome b(-245), which is a |
| | | component of the NADPH oxidase complex responsible |
| | | for the respiratory burst in phagocytes. |
| prion protein (PRNP) | AW452130.1 | |
| peptide methionine | NM_012331 | Oxidation of methionine residues in proteins is mediated |
| sulfoxide reductase | | by various biologic oxidants such as H(2)O(2), hydroxyl |
| (MSRA) | | radicals, hypochlorite, and superoxide ions. The oxidized |
| | | product, methionine sulfoxide, can be enzymatically |
| | | reduced back to methionine by peptide methionine |
| | | sulfoxide reductase (MSRA; EC 1.8.4.6) |
| glutathione peroxidase | NP_000572.2 | Cellular antioxidant enzymes such as glutathione |
| (GPX1) | | peroxidase-1 and superoxide dismutase have a central |
| | | role in control of reactive oxygen species. |
| thioredoxin-interacting | NM_006472 | TXNIP is a critical regulator of biomechanical signaling. |
| protein (TXNIP) | | |
| superoxide dismutase 2 | NM_000636 | The SOD2 gene encodes an intramitochondrial free |
| (SOD2) | | radical scavenging enzyme that is the first line of |
| | | defense against superoxide produced as a byproduct of |
| | | oxidative phosphorylation. |
| catalase (CAT) | NM_001752 | |
| xpa gene (XPA) | NM_000380 | The XPA gene encodes a protein involved in DNA |
| | | excision repair. |
| isocitrate dehydrogenase | NM_005896 | |
| 1 (IDH1) | | |
| glutathione peroxidase 4 | NM_002085 | Glutathione peroxidase-4 belongs to the family of |
| (GPX4) | | selenium-dependent peroxidases |

Methods are also provided for optimizing therapy, by first classification, and based on that information, selecting the appropriate therapy, dose, treatment modality, etc. which optimizes the differential between delivery of an anti-proliferative treatment to the undesirable target cells, while minimizing undesirable toxicity. The treatment is optimized by selection for a treatment that minimizes undesirable toxicity, while providing for effective anti-proliferative activity. The invention finds use in the prevention, treatment, detection or research of cancer, particularly carcinomas.

“Diagnosis” as used herein generally includes determination of a subject's susceptibility to a disease or disorder, determination as to whether a subject is presently affected by a disease or disorder, prognosis of a subject affected by a disease or disorder (e.g., identification of cancerous states, stages of cancer, or responsiveness of cancer to therapy), and use of therametrics (e.g., monitoring a subject's condition to provide information as to the effect or efficacy of therapy).

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

Clinical samples for use in the methods of the invention may be obtained from a variety of sources, particularly biopsy samples, although in some instances samples such as blood, bone marrow, lymph, cerebrospinal fluid, synovial fluid, and the like may be used. Such samples can be separated by centrifugation, elutriation, density gradient separation, apheresis, affinity selection, panning, FACS, centrifugation with Hypaque, etc. prior to analysis. Once a sample is obtained, it can be used directly, frozen, or maintained in appropriate culture medium for short periods of time. Various media can be employed to maintain cells. The samples may be obtained by any convenient procedure, such as the drawing of blood, venipuncture, biopsy, or the like. Usually a sample will comprise at least about 10² cells, more usually at least about 10³ cells, and preferable 10⁴, 10⁵ or more cells. Typically the samples will be from human patients, although animal models may find use, e.g. equine, bovine, porcine, canine, feline, rodent, e.g. mice, rats, hamster, primate, etc.

An appropriate solution may be used for dispersion or suspension of the cell sample. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Analysis of the cell staining will use conventional methods. Techniques providing accurate enumeration include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide).

Of particular interest is the use of antibodies as affinity reagents. Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The antibodies are added to a suspension of cells, and incubated for a period of time sufficient to bind the available cell surface antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated will be any medium that maintains the viability of the cells. A preferred medium is phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc.

The labeled cells are then quantitated as to the expression of cell surface markers as previously described.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans.

A “host cell”, as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

The terms “cancer”, “neoplasm”, and “tumor” are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancer stem cells is of particular interest. The term “normal” as used in the context of “normal cell,” is meant to refer to a cell of an untransformed phenotype or exhibiting a morphology of a non-transformed cell of the tissue type being examined. “Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

As used herein, cancer cells can be transformed but not tumorigenic. Tumorigenic cancer cells, i.e. cancer stem cells, have the ability to self-renew and to give rise to tumors when transplanted.

Of particular interest are the cancer stem cells of carcinomas. Carcinomas are cancers comprising neoplastic cells of epithelial origin. Epithelial cells cover the external surface of the body, line the internal cavities, and form the lining of glandular tissues. In adults, carcinomas are the most common forms of cancer. Carcinomas include the a variety of adenocarcinomas, for example in prostate, lung, etc.; adernocartical carcinoma; hepatocellular carcinoma; renal cell carcinoma, ovarian carcinoma, carcinoma in situ, ductal carcinoma, carcinoma of the breast, basal cell carcinoma; squamous cell carcinoma; transitional cell carcinoma; colon carcinoma; nasopharyngeal carcinoma; multilocular cystic renal cell carcinoma; oat cell carcinoma, large cell lung carcinoma; small cell lung carcinoma; etc. Carcinomas may be found in prostrate, pancreas, colon, brain (usually as secondary metastases), lung, breast, skin, etc.

Certain phenotypic attributes of carcinoma stem cells have been described in the art, and may include markers such as CD44, CD133, CD24, CD49f; ESA; CD166; and lineage panels. Lineage panels of interest include CD45, CD31, CD3, CD64, CD10, CD16, CD18, and GPA; CD45, CD31, CD140a, and Ter119; CD45, CD31 and CD140a. Typically lineage panels include at least one, at least two, at least three or more markers selected from CD45, CD31, CD3, CD64, CD10, CD16, CD18, GPA, CD140a and Ter119.

Examples of specific marker combinations and phenotypes are described, for example, by Al-Hajj et al. (2003) Prospective identification of tumorigenic breast cancer cells. Proc Natl Acad Sci USA 100, 3983-8; Singh et al. (2004) Identification of human brain tumour initiating cells. Nature 432, 396-401; Dalerba et al. (2007) Phenotypic characterization of human colorectal cancer stem cells. Proc Natl Acad Sci USA 104, 10158-63; O'Brien et al. (2006) A human colon cancer cell capable of initiating tumour growth in immunodeficient mice. Nature; Prince et al. (2007) Identification of a subpopulation of cells with cancer stem cell properties in head and neck squamous cell carcinoma. Proc Natl Acad Sci USA, each of which is herein specifically incorporated by reference for the teachings of cancer stem cell marker phenotypes. In some embodiments of the invention such phenotyping is used in conjunction with the detection of ROS species and related genes.

“Therapeutic target” refers to a gene or gene product that, upon modulation of its activity (e.g., by modulation of expression, biological activity, and the like), can provide for modulation of the cancerous phenotype. As used throughout, “modulation” is meant to refer to an increase or a decrease in the indicated phenomenon (e.g., modulation of a biological activity refers to an increase in a biological activity or a decrease in a biological activity).

### ROS Detection

In some embodiments of the invention, reactive oxygen species, or ROS, are detected in samples suspected of containing cancer stem cells. Such detection may be performed by in vivo imaging, or in an ex vivo sample. Where an ex vivo sample is used, it can be examined histologically, e.g. in a tissue section; or in a cell suspension, e.g. by flow cytometry. In any of such methods, a reagent, e.g. a detectable dye reactive with ROS such as DCF-DA, 2′,7′-dichlorodihydrofluorescein, DanePy, HO-1889NH, dihydrorhodamine-123, etc. is contacted with the suspected cancer stem cells. The change in dye resulting from ROS reaction is monitored, and usually compared to a control, e.g. a comparable non-tumorigenic cancer cell. Decreased levels of ROS is indicative that the CSC are resistant to radiation treatment and to certain chemotherapeutic agents, such as anthracyclines, including the compounds daunorubicin, adriamycin (doxorubicin) epirubicin, idarubicin, anamycin, MEN 10755, and the like.

Methods of quantitating ROS in vivo and in vitro are known in the art, for example as described by Tsatmali et al. (2006) Mol Cell Neurosci. 33(4): 345-357; Kunz et al. (2007) Photochem Photobiol Sci. 6(9):940; Rodriguez-Serrano et al. (2006) Plant Cell Environ. 29(8):1532-44; Hideg et al. (2002) Plant Cell Physiol. 43(10):1154-64; and Hanson et al. (2002) Photochem Photobiol. 76(1):57-63, each of which is herein specifically incorporated by reference.

In other embodiments of the invention, the cells are characterized for expression of one or more ROS-related genes, e.g. one or more of the genes set forth in Table 1, two or more, three or more, four or more, five or more, and up to all ten genes. By measured is meant qualitatively or quantitatively estimating the level of the gene product in a first biological sample either directly (e.g. by determining or estimating absolute levels of gene product) or relatively by comparing the levels to a second control biological sample. In some embodiments the second control biological sample is obtained from an individual not having cancer, in other embodiments the control is cancer cells of a similar type, but which are not CSC. As will be appreciated in the art, once a standard control level of gene expression is known, it can be used repeatedly as a standard for comparison. Detection of differential hybridization, when compared to a suitable control, is an indication of the presence in the sample of a polynucleotide that is differentially expressed in a cancer cell. Detection can also be accomplished by any known method, including, but not limited to, in situ hybridization, PCR (polymerase chain reaction), RT-PCR (reverse transcription-PCR), and “Northern” or RNA blotting, arrays, microarrays, etc, or combinations of such techniques, using a suitably labeled polynucleotide. A variety of labels and labeling methods for polynucleotides are known in the art and can be used in the assay methods of the invention. Specific hybridization can be determined by comparison to appropriate controls.

In some embodiments of the invention, the tumor sample is contacted with one or more known markers of cancer stem cells, particularly cell surface markers, and the level of ROS is determined for the CSC fraction of cells in the sample. Such markers of CSC are known in the art as described herein. In some embodiments flow cytometry is used to separate the CSC fraction from non-tumorigenic cells. In other embodiments that cells are histologically stained.

Samples, including tissue sections, slides, etc. containing cancer tissue may be frozen, embedded, present in a tissue microarray, suspended in media, and the like. The reagents, e.g. antibodies, polynucleotide probes, etc. may be detectably labeled, or may be indirectly labeled in the staining procedure.

Characterization of cancer stem cells with respect to ROS allows for the development of new treatments that are specifically targeted against this critical population of cells resulting in more effective therapies. In some embodiments of the invention, the number of ROS^{low} CSC in a patient sample is determined relative to the total number of cancer cells, where a greater percentage of ROS^{low} CSC is indicative of the radiation and drug resistance of the cancer. The quantitation of ROS^{low} CSC in a patient sample may be compared to a reference population, e.g. a patient sample such as a blood sample, a remission patient sample, etc. In some embodiments, the quantitation of ROS^{low} CSC is performed during the course of treatment, where the percentage cancer cells that are ROS^{low} CSC are quantitated before, during and as follow-up to a course of therapy. Desirably, therapy targeted to cancer stem cells results in a decrease in the total number, and/or percentage of CSC in a patient sample.

In one embodiment of the invention, a sample from a carcinoma patient is stained with reagents specific for ROS marker or combination of markers of the present invention. The analysis of staining patterns provides the relative distribution of ROS^{low} CSC, which distribution predicts the responsiveness of the cancer. In some embodiments, the sample is analyzed by histochemistry, including immunohistochemistry, in situ hybridization, and the like, for the presence of CD34⁺CD38⁻ cells that express a marker or combination of markers of the present invention. The presence of such cells indicates the presence of CSC.

In one embodiment, the patient sample is compared to a control, or a standard test value. In another embodiment, the patient sample is compared to a pre-leukemia sample, or to one or more time points through the course of the disease.

The information thus derived is useful in prognosis and diagnosis, including susceptibility to acceleration of disease, status of a diseased state and response to changes in the environment, such as the passage of time, treatment with drugs or other modalities. The cells can also be classified as to their ability to respond to therapeutic agents and treatments, isolated for research purposes, screened for gene expression, and the like. The clinical samples can be further characterized by genetic analysis, proteomics, cell surface staining, or other means, in order to determine the presence of markers that are useful in classification. For example, genetic abnormalities can be causative of disease susceptibility or drug responsiveness, or can be linked to such phenotypes.

The analysis obtained from a patient sample, and a reference analysis is accomplished by the use of suitable deduction protocols, AI systems, statistical comparisons, etc. A comparison with a reference differential progenitor analysis from normal cells, cells from similarly diseased tissue, and the like, can provide an indication of the disease staging. A database of reference differential progenitor analyses can be compiled. An analysis of particular interest tracks a patient, e.g. in the stages of disease, such that acceleration of disease is observed at an early stage. The methods of the invention allow early therapeutic intervention, e.g. initiation of chemotherapy, increase of chemotherapy dose, changing selection of chemotherapeutic drug, and the like.

### Polypeptide and Polynucleotide Sequences and Antibodies

The invention provides ROS; polynucleotides and polypeptides that represent genes that are differentially expressed in human CSC. These ROS; polynucleotides, polypeptides and fragments thereof have uses that include, but are not limited to, diagnostic probes and primers, as immunogens for antibodies useful in cancer diagnosis and therapy, and the like as discussed herein.

Nucleic acid compositions include fragments and primers, and are at least about 15 by in length, at least about 30 by in length, at least about 50 by in length, at least about 100 bp, at least about 200 by in length, at least about 300 by in length, at least about 500 by in length, at least about 800 by in length, at least about 1 kb in length, at least about 2.0 kb in length, at least about 3.0 kb in length, at least about 5 kb in length, at least about 10 kb in length, at least about 50 kb in length and are usually less than about 200 kb in length. In some embodiments, a fragment of a polynucleotide is the coding sequence of a polynucleotide. Also included are variants or degenerate variants of a sequence provided herein. In general, variants of a polynucleotide provided herein have a fragment of sequence identity that is greater than at least about 65%, greater than at least about 70%, greater than at least about 75%, greater than at least about 80%, greater than at least about 85%, or greater than at least about 90%, 95%, 96%, 97%, 98%, 99% or more (i.e. 100%) as compared to an identically sized fragment of a provided sequence. as determined by the Smith-Waterman homology search algorithm as implemented in MPSRCH program (Oxford Molecular). Nucleic acids having sequence similarity can be detected by hybridization under low stringency conditions, for example, at 50° C. and 10×SSC (0.9 M saline/0.09 M sodium citrate) and remain bound when subjected to washing at 55° C. in 1×SSC. Sequence identity can be determined by hybridization under high stringency conditions, for example, at 50° C. or higher and 0.1×SSC (9 mM saline/0.9 mM sodium citrate). Hybridization methods and conditions are well known in the art, see, e.g., U.S. Pat. No. 5,707,829. Nucleic acids that are substantially identical to the provided polynucleotide sequences, e.g. allelic variants, genetically altered versions of the gene, etc., bind to the provided polynucleotide sequences under stringent hybridization conditions.

Probes specific to the polynucleotides described herein can be generated using the polynucleotide sequences disclosed herein. The probes are usually a fragment of a polynucleotide sequences provided herein. The probes can be synthesized chemically or can be generated from longer polynucleotides using restriction enzymes. The probes can be labeled, for example, with a radioactive, biotinylated, or fluorescent tag. Preferably, probes are designed based upon an identifying sequence of any one of the polynucleotide sequences provided herein.

The polypeptides contemplated by the invention include those encoded by the disclosed polynucleotides and the genes to which these polynucleotides correspond, as well as nucleic acids that, by virtue of the degeneracy of the genetic code, are not identical in sequence to the disclosed polynucleotides. Further polypeptides contemplated by the invention include polypeptides that are encoded by polynucleotides that hybridize to polynucleotide of the sequence listing. Thus, the invention includes within its scope a polypeptide encoded by a polynucleotide having the sequence of any one of the polynucleotide sequences provided herein, or a variant thereof.

In general, the term “polypeptide” as used herein refers to both the full length polypeptide encoded by the recited polynucleotide, the polypeptide encoded by the gene represented by the recited polynucleotide, as well as portions or fragments thereof. “Polypeptides” also includes variants of the naturally occurring proteins, where such variants are homologous or substantially similar to the naturally occurring protein, and can be of an origin of the same or different species as the naturally occurring protein. In general, variant polypeptides have a sequence that has at least about 80%, usually at least about 90%, and more usually at least about 98% sequence identity with a differentially expressed polypeptide described herein. The variant polypeptides can be naturally or non-naturally glycosylated, i.e., the polypeptide has a glycosylation pattern that differs from the glycosylation pattern found in the corresponding naturally occurring protein.

Fragments of the polypeptides disclosed herein, particularly biologically active fragments and/or fragments corresponding to functional domains, are of interest. Fragments of interest will typically be at least about 10 aa to at least about 15 aa in length, usually at least about 50 aa in length, and can be as long as 300 aa in length or longer, but will usually not exceed about 1000 aa in length, where the fragment will have a stretch of amino acids that is identical to a polypeptide encoded by a polynucleotide having a sequence of any one of the polynucleotide sequences provided herein, or a homolog thereof. A fragment “at least 20 aa in length,” for example, is intended to include 20 or more contiguous amino acids from, for example, the polypeptide encoded by a cDNA, in a cDNA clone contained in a deposited library or the complementary stand thereof. In this context “about” includes the particularly recited value or a value larger or smaller by several (5, 4, 3, 2, or 1) amino acids. The protein variants described herein are encoded by polynucleotides that are within the scope of the invention. The genetic code can be used to select the appropriate codons to construct the corresponding variants. The polynucleotides may be used to produce polypeptides, and these polypeptides may be used to produce antibodies by known methods described above and below.

A polypeptide of this invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast higher plant, insect, and mammalian cells.

Gene products, including polypeptides, mRNA (particularly mRNAs having distinct secondary and/or tertiary structures), cDNA, or complete gene, can be prepared and used for raising antibodies for experimental, diagnostic, and therapeutic purposes. Antibodies may be used to identify CSC cells or subtypes. The polynucleotide or related cDNA is expressed as described herein, and antibodies are prepared. These antibodies are specific to an epitope on the polypeptide encoded by the polynucleotide, and can precipitate or bind to the corresponding native protein in a cell or tissue preparation or in a cell-free extract of an in vitro expression system.

The antibodies may be utilized for immunophenotyping of cells and biological samples. The translation product of a differentially expressed gene may be useful as a marker. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al. Cell, 96:737-49 (1999)). These techniques allow for the screening of particular populations of cells; in immunohistochemistry of biopsy samples; in detecting the presence of markers shed by cancer cells into the blood and other biologic fluids, and the like.

### Screening Assays

CSC expressing a marker or combination of markers of the present invention are also useful for in vitro assays and screening to detect factors and chemotherapeutic agents that are active on cancer stem cells, and that alter the level of ROS in the CSC. Of particular interest are screening assays for agents that are active on human cells. A wide variety of assays may be used for this purpose, including immunoassays for protein binding; determination of cell growth, differentiation and functional activity; production of factors; and the like. In other embodiments, isolated polypeptides corresponding to a marker or combination of markers of the present invention are useful in drug screening assays.

In screening assays for biologically active agents, anti-proliferative drugs, etc. the marker or CSC composition is contacted with the agent of interest, and the effect of the agent assessed by monitoring ROS parameters on cells, such as expression of ROS-related genes, direct quantitation of ROS, and the like. The cells may be freshly isolated, cultured, genetically altered, and the like. The cells may be environmentally induced variants of clonal cultures: e.g. split into independent cultures and grown under distinct conditions, for example with or without drugs; in the presence or absence of cytokines or combinations thereof. The manner in which cells respond to an agent, particularly a pharmacologic agent, including the timing of responses, is an important reflection of the physiologic state of the cell.

While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

In addition to complex biological agents candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term “samples” also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable ROS detection, usually from about 0.1 to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cell samples, usually in conjunction with cells lacking the agent. The change in ROS parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of the selected markers. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

Kits may be provided, where the kit will comprise staining reagents that are sufficient to differentially identify the CSC described herein. A combination of interest may include one or more reagents specific for a marker or combination of markers of the present invention, and may further include an ROS reactive dye. The staining reagents are preferably antibodies, and may be detectably labeled. Kits may also include tubes, buffers, etc., and instructions for use.

Each publication cited in this specification is hereby incorporated by reference in its entirety for all purposes.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

### EXPERIMENTAL

### Example 1

### Association of Reactive Oxygen Species Levels and Radioresistance in Cancer Stem Cells

Metabolism of oxygen, while central to life, also produces reactive oxygen species (ROS) that have been implicated in processes as diverse as cancer, cardiovascular disease, and aging. It has recently been shown that central nervous system stem cells and hematopoietic stem cells and early progenitors contain lower levels of ROS than their more mature progeny and that these differences appear to be critical for maintaining stem cell function. We hypothesized that epithelial tissue stem cells and their cancer stem cell (CSC) counterparts may also share this property.

Here we show that normal mammary epithelial stem cells contain lower concentrations of ROS than their more mature progeny cells. Congruently, subsets of CSCs in some human and murine breast tumors contain lower ROS levels than corresponding non-tumorigenic cells (NTCs). Consistent with ROS being critical mediators of ionizing radiation-induced cell killing, CSCs in these tumors develop less DNA damage and are preferentially spared after irradiation compared to NTCs. Lower ROS levels in CSCs are associated with increased expression of free radical scavenging systems. Pharmacologic depletion of ROS scavengers in CSCs significantly decreases their clonogenicity and results in radiosensitization. These results indicate that, similar to normal tissue stem cells, subsets of CSCs in some tumors contain lower ROS levels and enhanced ROS defenses compared to their non-tumorigenic progeny, which may contribute to tumor radioresistance.

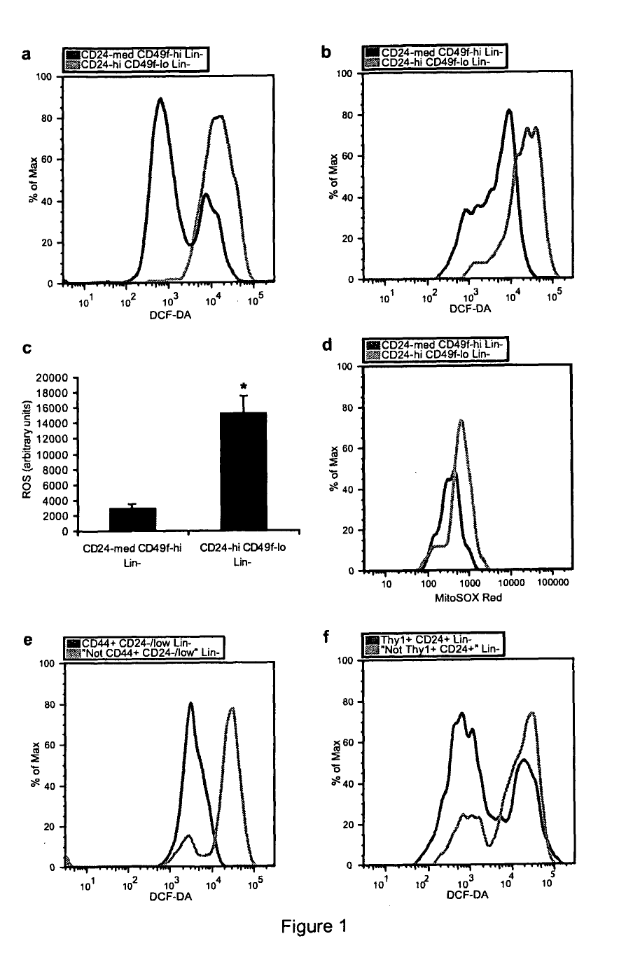

We began by asking whether low ROS concentrations that appear to be critical to self renewal of hematopoietic stem cells (HSCs) are also a property of mammary epithelial stem cells by isolating CD24^{med}CD49f^{high}Lin⁻ mammary cells, a population enriched for mammary repopulating units (MRUs), where lineage markers included CD45, CD31, CD140a, and Ter119, and b) Thy1/CD24, and CD24^{high}CD49f^{low}Lin⁻ progenitor cells, where lineage markers included CD45, CD31, and CD140a, by flow cytometry and measuring intracellular concentrations of prooxidants using 2′-7′-dichlorofluorescein diacetate (DCF-DA) staining. Cells in the MRU-enriched population contained significantly lower concentrations of ROS than the progenitor-enriched cells in two different strains of mice (FIG. 1a-c). Specifically, the MRU-enriched populations displayed low to intermediate ROS levels, while the progenitor-enriched populations contained more uniformly high levels of ROS. Similarly, analysis of the two populations with MitoSOX Red, a highly selective detection method for mitochondrial superoxide, revealed lower superoxide levels in the MRU-enriched population (FIG. 1d). In order to assess if mammary repopulating activity was related to intracellular concentrations of ROS, we transplanted CD24^{med}CD49f^{high}Lin⁻ cells based on their levels of DCFDA staining. Mammary stem cells with both low and intermediate ROS levels gave rise to epithelial outgrowths when transplanted into cleared fat pads (Table 1). Similar heterogeneity of ROS concentrations was recently demonstrated in HSC-enriched populations where it may have functional significance in modulating the HSC-niche interaction. Given the conservation of low ROS levels in several types of normal tissue stem cells, we hypothesized that CSCs in some tumors may also contain lower concentrations of ROS than their non-tumorigenic progeny.

In order to investigate ROS biology in human CSCs, we began by examining the expression of genes involved in ROS metabolism in primary human breast CSCs and NTCs. Using microarray data from human breast CSC-enriched populations and NTCs and a curated list of genes involved in ROS metabolism (see methods), Gene Set Enrichment Analysis (GSEA) revealed that the expression of ROS genes was highly overrepresented in the CD44⁺CD24^{-/low}Lin⁻ breast CSC-enriched population compared to NTCs (p<0.001). Murine Thy1⁺CD24⁺Lin⁻ cancer cells and “Not Thy1⁺CD24⁺” Lin⁻ non-tumorigenic cells were isolated from MMTV-Wnt-1 breast tumors by flow cytometry and ROS levels were analyzed as in FIG. 1. The percentage of cells with low ROS concentrations was quantified within the two populations. Mean±s.e.m. (n=7; p=0.003). The ROS genes identified as the core enriched genes by GSEA included a number of important antioxidant genes (Table 2). Thus, gene expression profiles of human breast CSC-containing populations suggest that they contain higher levels of antioxidant defense systems than NTCs.

Next, we directly assessed ROS levels in human tumor subpopulations. To do this the CD44⁺CD24^{-/low}Lin⁻ breast CSC-enriched population and the corresponding “Not CD44⁺CD24^{-/low}” Lin⁻ NTC population were purified from surgically resected breast tumors. Forward scatter area versus forward scatter width profiles were used to eliminate cell doublets and dead cells were eliminated by excluding DAPI⁺ cells. Lineage positive cells were excluded using CD45, CD3, CD10, CD64, CD31, and Glycophorin A. CD44⁺CD24^{-/low}Lin⁻ cells and non-tumorigenic cells were sorted using the indicated gates. DCF-DA staining revealed that the CSC-enriched population in the human breast tumors we examined contained significantly lower levels of prooxidants than the NTC population. In some breast tumors, the vast majority of cells in the CSC containing fraction displayed a low ROS phenotype compared to NTCs (FIG. 1e) while in others it was restricted to a significant subset of CSCs. CD44⁺CD24^{-/low}Lin⁻ cells (cancer stem cell-enriched population) and “Not CD44⁺CD24^{-/low}” Lin⁻ non-tumorigenic cells were isolated using flow cytometry. Lineage positive cells were excluded using CD45, CD31, CD3, CD64, CD10, CD16, CD18, and GPA. Intracellular ROS concentrations were subsequently measured by DCF-DA staining. We found a similar enrichment of cells with low ROS concentrations in a head and neck tumor. Thus, CSC-enriched populations from some human tumors contain lower average intracellular ROS levels than corresponding NTC populations.

Recently, we have demonstrated that Thy1⁺CD24⁺Lin⁻ cells in the majority of spontaneously developing breast tumors from MMTV-Wnt-1 mice are highly enriched for tumorigenic activity. We therefore asked whether the CSC-enriched population in this model system also displays a low ROS phenotype. Thy1⁺CD24⁺Lin⁻ cells and “Not Thy1⁺CD24⁺” Lin⁻ non-tumorigenic cells from MMTV-Wnt-1 breast tumors that were irradiated in vitro in bulk with 1 Gy of IR. The two populations were then sorted using flow cytometry and the resulting cells were immunostained for γ-H2AX. The percentage of cells with nuclear foci was quantified.

ROS analysis using DCF-DA revealed that in tumors in which the Thy1⁺CD24⁺Lin⁻ population was enriched for CSCs, this population contained a significantly higher fraction of cells with low prooxidant levels than the “Not Thy1⁺CD24⁺” Lin⁻ non-tumorigenic population (FIG. 1f). Thy1⁺CD24⁺Lin⁻ cells contained two main sub-populations of cells based on ROS concentration, with the low ROS subpopulation being significantly overrepresented compared to NTCs. In order to confirm the presence of CSCs within the low ROS subpopulation, we transplanted CSCs based on their DCF-DA staining and found that both the low and high ROS subsets of Thy1⁺CD24⁺Lin⁻ cells gave rise to tumors in recipient animals (Table 3). Thus, a subset of CSCs from these MMTV-Wnt-1 tumors displayed low baseline levels of ROS compared to NTCs.

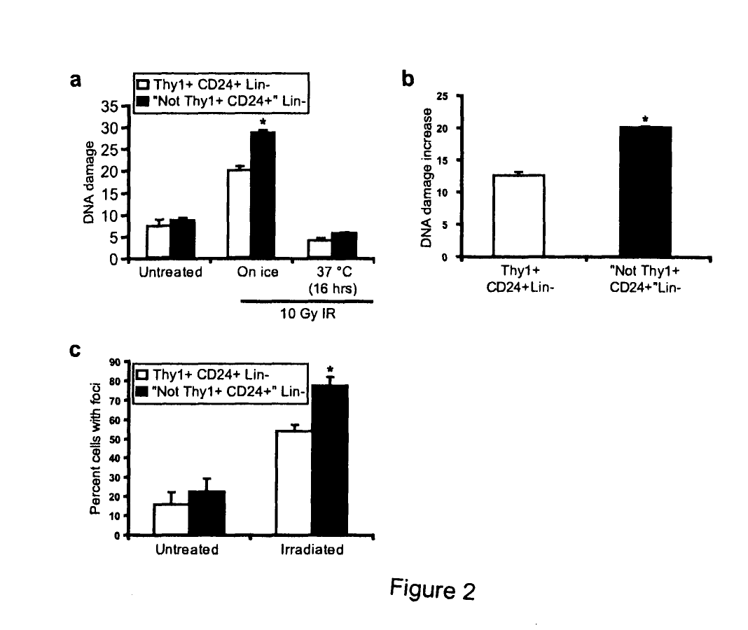

It is well established that cell killing after exposure to ionizing radiation (IR) and a subset of cytotoxic chemotherapeutics is partially mediated by free radicals. Given our observations of increased expression of ROS defense genes in CSCs, we were therefore interested in testing whether CSC-enriched populations develop less DNA damage after IR than NTCs. In order to examine DNA damage immediately after irradiation, we purified Thy1⁺CD24⁺Lin⁻ cells and NTCs from MMTV-Wnt-1 tumors by flow cytometry and irradiated them on ice. Cells were then either left on ice or incubated at 37° C., before being analyzed using the alkaline comet assay. While untreated cells did not display significantly different levels of DNA damage, there were fewer DNA-strand breaks in the Thy1⁺CD24⁺Lin⁻ cells than NTCs immediately after exposure to IR (FIG. 2a-b). These findings are consistent with the hypothesis that enhanced expression of ROS defenses in CSCs contributes to reduced levels of DNA damage after irradiation.

Since the alkaline comet assay mainly measures single strand breaks and since double strand breaks are important for IR-induced lethality, we also analyzed levels of double strand breaks as reflected by phosphorylated histone 2AX (H2AX) nuclear foci after in vitro irradiation. As with the comet assay, we again observed significantly lower levels of DNA damage in Thy1⁺CD24⁺Lin⁻ CSC-enriched cells than in NTCs. The cell cycle status of Thy1⁺CD24⁺Lin⁻ cells was determined by 7AAD staining of DNA content.

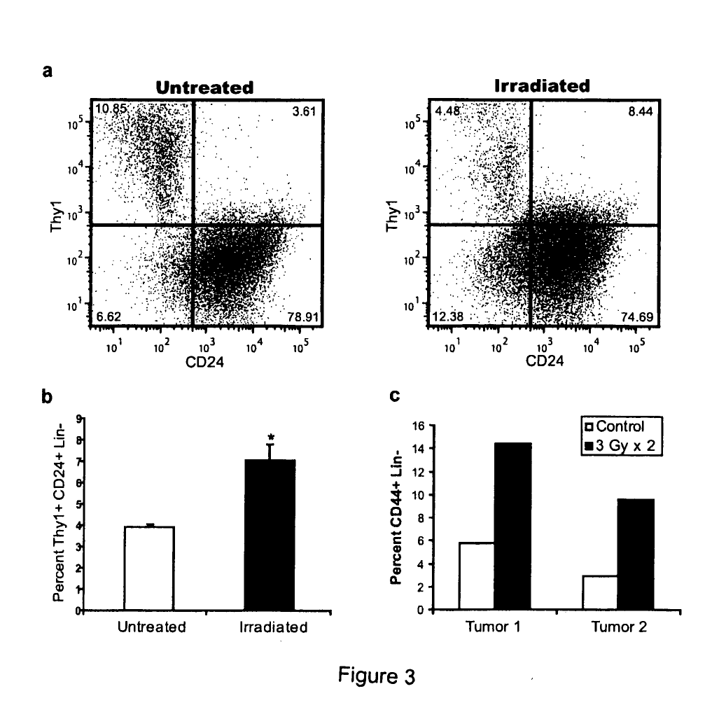

We also measured phosphorylated H2AX foci after in vivo irradiation of MMTV-Wnt-1 tumors, and again found that Thy1⁺CD24⁺Lin″ cells contained fewer foci than NTCs (FIG. 2c). Thus, consistent with their lower baseline levels of ROS, Thy1⁺CD24⁺Lin″ CSC enriched cells isolated from these tumors developed less DNA strand breaks than NTCs after exposure to IR. Given these findings, CSCs would be expected to preferentially survive exposure to IR in intact tumors. Mice bearing MMTV-Wnt-1 tumors were therefore treated with short, fractionated courses of IR and the percentage of the Thy1⁺CD24⁺Lin″ CSC-enriched population before and after irradiation was analyzed using flow cytometry. On average, we found an approximately 2 fold increase in the percentage of the Thy1⁺CD24⁺Lin⁻ CSC-enriched population compared with “Not Thy1⁺CD24⁺” Lin⁻ NTCs in the irradiated tumors, suggesting that CSCs are relatively radioresistant compared with NTCs (FIG. 3a-b). We found a similar increase in the fraction of CD44⁺Lin⁻ CSC-enriched population when we irradiated human head and neck cancer xenografts grown in immunodeficient mice (FIG. 3c). Other investigators have documented similar radioresistance of CSCs in brain tumors and a breast cancer cell line. Thus, CSCs in some murine and human tumors are relatively radioresistant compared to their NTC counterparts.

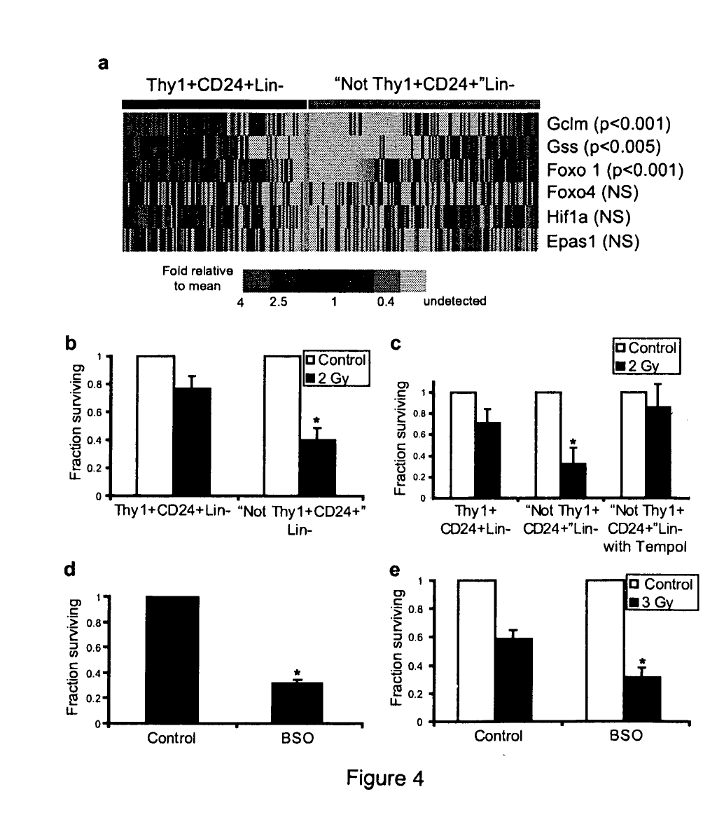

Since a significant fraction of murine breast CSCs contained relatively low levels of ROS, we hypothesized that these cells may express enhanced levels of ROS defenses compared to their NTC counterparts. We were particularly interested in glutathione (GSH), a critical cellular reducing agent and antioxidant which has been implicated in chemotherapy and radiotherapy resistance of cancer cells. Since our prior analyses revealed heterogeneity within CSC enriched populations and since single cell gene expression studies have revealed significant variation in gene expression in other stem cell populations, we investigated expression of critical GSH biosynthesis genes in MMTV-Wnt-1 CSCs and NTCs using single cell qRT-PCR. This analysis revealed significant overexpression of Gc/m (p<0.001) and Gss (p<0.001) in a large fraction of cells within the CSC-enriched population, the former of which encodes the regulatory subunit of the enzyme (glutamate-cysteine ligase) that catalyzes the rate limiting step of GSH synthesis (FIG. 4a). Furthermore, Foxo1, a transcription factor implicated in the regulation of an anti-ROS gene expression program in HSCs5, was also overexpressed in CSCs compared to NTCs (p<0.001; FIG. 4a). Other genes, including Hifla, Epas1, and Foxo4 were not differentially expressed. Thus, genes controlling GSH biosynthesis were overexpressed by many cells within the CSC-enriched population isolated from this tumor.

In order to pharmacologically manipulate ROS levels separately in CSCs and NTCs, we employed in vitro culture conditions that allowed both cell populations to produce colonies upon co-culture with irradiated feeder cells. We found that Thy1⁺CD24⁺Lin″ CSC-enriched cells were relatively radioresistant compared with NTCs (FIG. 4b and FIG. 11). When exposed to 2 Gy, a dose commonly administered clinically during daily treatments of breast cancer patients, 2.0-fold+/−0.2 more CSC-enriched colonies survived than NTC colonies. Next, we attempted to radioprotect NTCs by exposing them to the nitroxide antioxidant tempol. Pretreatment with tempol radioprotected NTCs, and resulted in survival levels similar to those seen in the CSC-enriched population (FIG. 4c).

Given the overexpression of genes involved in GSH synthesis by CSCs, we wished to assess the sensitivity of these cells to ROS elevation via pharmacologic depletion of GSH. Exposure of Thy1⁺CD24⁺Lin″ CSC-enriched cells to Buthionine Sulfoximine (BSO), which inhibits glutamate-cysteine ligase, decreased their colony forming ability by approximately three fold (FIG. 4d). Finally, we asked if GSH depletion would radiosensitize CSCs. As shown in FIG. 4e, BSO pretreatment of Thy1⁺CD24⁺Lin⁻ CSC-enriched cells led to significant radiosensitization. These data demonstrate the importance of low ROS levels and antioxidant defenses to CSC survival and radiosensitivity in these tumors. Our data indicate that normal breast stem cells and a subset of CSCs in some tumors arising in both mice and humans contain lower levels of ROS than their cellular descendants. Taken together with previous reports of low ROS concentrations in other normal tissue stem cells, these findings suggest that stem cells in diverse systems have conserved this attribute, which likely helps to protect their genomes from endogenous and exogenous ROS-mediated damage. The mechanism leading to low ROS levels in some CSCs appears to be at least partially due to the increased production of free radical scavengers. Notably, there appears to be significant heterogeneity of ROS levels in both normal stem cell and CSC-enriched populations, which could reflect that the enriched populations contain both stem and non-stem cells and/or that ROS levels within stem cells can differ based on environmental factors that alter the balance of endogenous production and the expression of scavenging pathways. The low ROS subset found in the various stem cell populations may also represent a quiescent subpopulation. Heterogeneity of ROS levels may influence the extent to which CSC-enriched populations are resistant to therapies such as ionizing radiation.

In light of recent findings that CSCs in glioblastoma multiforme display enhanced DNA repair capabilities, it appears that CSCs may resist standard cytotoxic therapies through a combination of mechanisms, and that these may be unique to a given tumor. In the case of human CSCs, the frequency with which these cells display low ROS levels or enhanced DNA repair remains to be determined, particularly since the normal transformation precursor may be either stem or progenitor cells and since ROS concentration (FIGS. 1 and 5) and increased DNA repair capabilities appear to partially reflect differentiation state. Clinical therapies could likely be optimized by patient- and tumor-specific identification of CSC-resistance mechanisms and overcoming low ROS levels within CSCs may be a useful method for improving local and systemic oncologic therapies.

### Methods Summary

Cells were analyzed, collected by FACS, and injected into recipient mice as described with minor modifications from mouse mammary glands, human breast cancers, human head and neck cancers, and MMTV-Wnt-1 mouse tumors. For human samples, informed consent was obtained after approval of protocols by the Stanford University and City of Hope Institutional Review Boards. For intracellular ROS analysis, cells were loaded with 10 μM DCF-DA (Invitrogen), incubated at 37° C. for 30 min, and immediately analyzed by flow cytometry. Cells were re-sorted based on their level of DCF-DA staining for transplant experiments. For MitoSOX Red experiments, cells were loaded with 5 μM MitoSOX Red at 37° C. for 20 min. DNA damage was evaluated using the single-cell gel electrophoresis assay under alkaline conditions. For γ-H2AX immunostaining, purified cells were cytospun onto poly-L-lysine coated slides, fixed, permeabilized, and stained with a phospho-specific (Ser 139) histone H2AX antibody (Cell Signaling Technology) followed by a secondary Alexa Fluor 488-conjugated antibody (Invitrogen). For single cell gene expression analysis, cells were sorted into 96 well plates containing CellsDirect qRT-PCR mix (Invitrogen). After reverse transcription, genes were pre-amplified (22 cycles) using the same Taqman primers (Applied Biosystems) used for quantification. Products were analyzed using qPCR DynamicArray microfluidic chips (Fluidigm). For in vitro colony assays, cells were cultured in Epicult B medium (StemCell Technologies) with 5% serum in the presence of ˜13,000 cm−2 irradiated NIH-3T3 cells. After 24-48 hrs, the media was replaced with serum-free Epicult B, and colonies counted ˜7 days later. GSEA14 was performed using previously published microarray data of CSCs and NTCs from primary breast tumor samples and a curated list of ROS genes (Table 4). Levels of significance were determined by Student's t-tests using (=0.05.

Murine mammary stem cell isolation. Mammary glands from 6-12 week old female C57BI/6J or 129S1/SvImJ mice were dissociated as described with minor modifications. Specifically, mammary fat pads were harvested and placed directly into Medium 199 (Gibco BRL) supplemented with 20 mM HEPES and Penicillin, Streptomycin, and Actinomycin (PSA). Tissue was minced using sterile razor blades and 4 Wünsch units of Liberase Blendzyme 4 (Roche 1988476) and 100 Kunitz units of DNase I (Sigma D4263) were added. Tissue was incubated for 60-90 minutes in a 37° C./5% CO₂ incubator, during which the cells were mechanically aspirated every 30 minutes. Cells were pelleted by centrifugation for 5 min at 4° C. and 350×g. After lysis of the red blood cells with ACK lysis buffer (Gibco), a single cell suspension was obtained by further enzymatic digestion for ˜2 min in 0.25% trypsin, followed by another ˜2 min in 5 mg/ml dispase II (StemCell Technologies) plus 200 Kunitz units DNase I (Sigma). Cells were then filtered through 40-m nylon mesh, pelleted, and resuspended in staining media (HBSS+2% HICS). Cells were counted using trypan blue dye exclusion.

Tumor dissociation. Human and mouse tumors were dissociated as previously described with minor modifications. Depending on the time of the surgical case, some of the human tumors were kept over night at 4° C. prior to dissociation. Tumors from patients or MMTV-Wnt-1 tumor-bearing FVB/NJ female mice were minced with a razor blade and suspended in 20 ml of Medium 199 (Gibco BRL) supplemented with 20 mM Hepes. The dissociation enzyme cocktail consisted of 100 Kunitz units of DNAse I (Sigma D4263), 8 Wunsch units of Liberase Blendzyme 2 (Roche 1998433), and 8 Wunsch units Liberase Blendzyme 4 (Roche 1988476). Tumors were digested to completion (1.5-2.5 hours at 37° C./5% CO₂) with pipetting every 30 minutes for manual dissociation. Once digested, 30 ml of RPMI (BioWhittaker) with 10% calf serum (HICS) was added to the digestion solution to inactivate the collagenases. A 40 μm nylon filter was used to filter the sample. After pelleting, cells were resuspended in 5 ml of ACK buffer for red blood cell lysis. HBSS (BioWhittaker) with 2% heat-inactivated calf serum (HICS) was used to dilute the ACK buffer and the cells were again filtered through a 40 μm nylon filter. The filtered cells were spun down and resuspended in HBSS with 2% HICS.

Cell staining and flow cytometry. Cells were stained at a concentration of 1×10⁶ cells per 100 μL of HBSS with 2% HICS (staining media). Cells were blocked with rabbit or mouse IgG (1 mg/ml) at 1:100 dilution and antibodies were added at appropriate dilutions determined from titering experiments. For the normal mammary stem cell experiments, antibodies included CD49f, CD31, CD45, Ter119 (BD Pharmingen), CD24, Thy1.2, and CD140a (eBioscience). For the murine MMTV-Wnt-1 breast cancer experiments, antibodies included CD24, Thy1.1, CD140a (eBioscience), CD45 and CD31 (BD Pharmingen). For the human breast cancer experiments, antibodies included CD44, CD24, CD45, CD3, CD20, CD10, Glycophorin A (BD Pharmingen), CD31 (eBioscience) and CD64 (Dako). For the primary human head and neck cancer experiment, antibodies included CD44, CD45 (BD Pharmingen), and CD31 (eBioscience) and for the xenograft experiments, antibodies included CD44, mCD45 (BD Pharmingen), mCD31 (Abcam), H-2 K_{d} (eBioscience), mCD45. Cells were stained for 20 minutes on ice and washed with staining media. When biotinylated primary antibodies were used, cells were additionally stained with streptavidin-conjugated fluorophores and washed. Ultimately, cells were resuspended in staining media containing 7-aminoactinomycin D (7-AAD, 1 μg/ml final concentration) or 4′-6-Diamidino-2-phenylindole (DAPI, 1 μg/ml final concentration) to stain dead cells. For all experiments, cells were analyzed and sorted using a FACSAria cell sorter (BD Bioscience). Side scatter and forward scatter profiles were used to eliminate debris and cell doublets. Dead cells were eliminated by excluding DAPI+ cells, whereas contaminating human or mouse Lin⁺ cells were eliminated by excluding cells labeled with the fluorophore used for the lineage antibody cocktail. In cell-sorting experiments, cell populations underwent two consecutive rounds of purification (double sorting) when the initial purity was not deemed high enough and a sufficient number of cells were available. Final purities ranged from −60% to >95%.

MMTV-Wnt-1 breast tumor radiation experiments. For in vivo irradiation experiments, tumor-bearing animals were irradiated on consecutive days with the indicated doses of ionizing radiation using a 160 kVp cabinet irradiator (Faxitron). Tumors were grown on the ventral surface of mice in the vicinity of the second mammary fat pad and the delivered dose was adjusted to compensate for attenuation by overlying tissues. Thirty-six hours after the final fraction was delivered, tumors were harvested, dissociated, and analyzed by flow cytometry as above. For each experiment, at least one untreated control tumor was analyzed in parallel. When multiple control tumors were available, the percentage of Thy1⁺CD24⁺Lin″ cells in these tumors were averaged to define the baseline percentage of Thy1⁺CD24⁺Lin″ cells for that experiment. In other experiments, tumors were irradiated and these tumors along with controls were harvested 15 minutes after irradiation. Tumors were dissociated as above and cells were stained with a phospho-specific (Ser 139) histone H2AX antibody (Cell Signaling Technology).

Human breast and head and neck cancer primary specimens. Primary tumor specimens were obtained with informed consent after approval of protocols by the Stanford University and City of Hope Institutional Review Boards. Tumors were from untreated patients, except for the two breast cancer tumors shown in FIG. 6a-d, which had been treated with neoadjuvant chemotherapy prior to resection. Human head and neck cancer xenograft radiation experiments. Tumors were grown subcutaneously on the backs of Rag2γDKO mice as previously described. Mice were irradiated as above, except that for each fraction half of the dose was delivered from the left side of the animal and half from the right. The non-tumor-bearing portions of each animal were shielded using custom made lead chambers.

MMTV-Wnt-1 DCF-DA transplant experiments. Lin⁻ or Thy1⁺CD24⁺Lin⁻ cells from tumors were sorted as described above. Cells were loaded with 10 μM DCF-DA (Invitrogen), incubated at 37° C. for 30 min, and sorted into “ROS-low” and “ROS-high” sub-populations based on their DCF-DA staining profile. Sorted cells were injected into FVB female mice in Matrigel (BD Bioscience) in the vicinity of the second mammary fat pad at the indicated cell numbers.

Normal mammary stem cell DCF-DA transplant experiments. CD24^{med}CD49f^{high}Lin⁻ mammary cells (enriched for mammary repopulating units) were isolated from mammary fat pads from C57BI/6J female mice as described above. Cells were loaded with 10 μM DCF-DA (Invitrogen), incubated at 37° C. for 30 min, and sorted into “ROS-low” and “ROS-mid” sub-populations based on their DCF-DA staining profile (in comparison to that of CD24^{high}CD49f^{low}Lin⁻ progenitor cells, which displayed a “ROS-high” profile). Mammary glands of 21-day-old female C57BI/6J mice were cleared of endogenous epithelium as previously described, and sorted cells were injected into each cleared fat pad using a Hamilton syringe. Injected glands were removed for whole mount analysis after 5-6 weeks. Transplants were scored as positive if epithelial structures consisting of ducts arising from a central point, with lobules and/or terminal end buds present.

In vitro colony assay. Sorted tumor cells were cultured in Epicult B medium (StemCell Technologies) with 5% serum in the presence of ˜13,000 cm⁻² irradiated NIH-3T3 cells. After 24-48 hrs, the media was replaced with serum-free Epicult B, and ˜7 days later, colonies were fixed with acetone:methanol (1:1), stained with Giemsa, and counted. Colonies were counted if they contained >=˜30 cells and the number of colonies in control wells was in the range of 30-100, depending on how many cells were sorted and the plating efficiency of a given tumor. For the L-S,R-Buthionine Sulfoximine (BSO) experiments, cells were cultured for 24 hours in modified mammosphere medium in the presence or absence of 1 mM BSO and the drug was removed immediately prior to irradiation. For the tempol experiments, cells were treated with 10 mM tempol for 15 minutes prior to irradiation, after which the drug was removed. For the rotenone experiments, cells were cultured for 24 hours in modified mammosphere medium in the presence or absence of 25 nM rotenone and the drug was removed immediately prior to irradiation. For all drug experiments, “drug only” controls were run in parallel to adjust for effects of each drug on baseline colony counts.

Gene Set Enrichment Analysis. To define a list of genes involved in ROS metabolism and regulation, we began with a previously generated list from a recently published study by Tothova et al. Briefly, this list was initially generated by these authors using the Gene Ontology GO:TERMFINDER program to classify genes by biological process, molecular function, or cellular component. The biological process terms included were: response to oxidative stress; response to reactive oxygen species; response to hydrogen peroxide; response to oxygen radical; and response to superoxide. We manually curated this list by performing PubMed based literature searches for each gene and only retaining those that had published evidence of involvement in ROS metabolism or regulation (i.e. removing genes that appeared to be included solely due to electronic curation with inferred evidence). This trimmed the ROS gene list from 55 to 36 unique symbols (Table 4). Gene Set Enrichment Analysis (GSEA) was then applied as previously described. The “core enriched genes” shown in FIG. 4 were defined by the algorithm.

Single cell gene expression analysis. For the single cell gene expression experiments we used qPCR DynamicArray microfluidic chips (Fluidigm). Single MMTV-Wnt-1 Thy1⁺CD24⁺Lin⁻ CSC-enriched cells (TG) and “Not Thy1⁺CD24⁺” Lin⁻ non-tumorigenic cells (NTG) cells were sorted by FACS into 96 well plates containing PCR mix (CellsDirect, Invitrogen) and RNase Inhibitor (Superaseln, Invitrogen). After hypotonic lysis we added RT-qPCR enzymes (SuperScript III RT/Platinum Taq, Invitrogen), and a mixture containing a pool of low concentration assays (primers/probes) for the genes of interest (Gclm-Mm00514996_m1, Gss-Mm00515065_m1, Foxo1-Mm00490672_m1, Foxo4-Mm00840140_g1, Hif1a-Mm00468875_m1, Epas1-Mm00438717_m1). Reverse transcription (15 minutes at 50° C., 2 minutes of 95° C.) was followed by pre-amplification for 22 PCR cycles (each cycle: 15 sec at 95° C., 4 minutes at 60° C.). Total RNA controls were run in parallel. The resulting amplified cDNA from each one of the cells was inserted into the chip sample inlets with Taqman qPCR mix (Applied Biosystems). Individual assays (primers/probes) were inserted into the chip assay inlets (2 replicates for each). The chip was loaded for one hour in a chip loader (Nanoflex, Fluidigm) and then transferred to a reader (Biomark, Fluidigm) for thermocycling and fluorescent quantification. To remove low quality gene assays, we discarded gene assays whose qPCR curves showed non-exponential increases. To remove low quality cells (e.g. dead cells) we discarded cells that did not express the housekeeping genes Actb (beta-actin) and Hprt1 (hypoxanthine guanine phosphoribosyl transferase 1). This resulted in a single cell gene expression dataset consisting of 248 cells (109 tumorigenic and 139 non-tumorigenic) from a total of 7 chip-runs. A two sample Kolmogorov-Smirnov (K-S) statistic was calculated to test if genes were differentially expressed in the two populations. We generated p values by permuting the sample labels (i.e. TG vs NTG) and comparing the actual K-S statistic to those in the permutation-derived null distribution. The p values were further corrected by Bonferroni correction to adjust for multiple hypothesis testing.

### Example 2

### Analysis of Cancer Stem Cells to Predict Response to Radiation

In view of evidence that cancer stem cells drive tumor development, it was predicted that resistance of the tumorigenic cancer cells would contribute to relapse after cytoxic therapy. This prediction has now been borne out. Compared to non-tumorigenic cancer cells, cancer stem cells are relatively resistant to radiation and chemotherapy, at least partly as a result of activation of pathways used to protect normal stem cells from damage from toxins. Some of the mechanisms that confer resistance to cytotoxic therapies to CSCs appear to also confer resistance of normal stem cells to these agents. These properties of CSC can provide the basis for prognostic and screening methods.

In approximately 50% of tumors examined to date, the CSCs (from epithelial cancers of the breast, colon and head and neck) contain significantly lower levels of ROS compared to their non-tumorigenic counterparts. This is associated with expression of gene pathways that modulate ROS production and/or scavenging. Radiation therapy may not sterilize tumors whose CSCs contain low levels of ROS.

Alternative tests can be used to measure ROS pathways in carcinoma stem cells (CSCs). In one embodiment, ROS are directly measured in the CSCs. To do this endoscopic biopsies of a patient's tumor are obtained. Flow cytometry can be used to isolate CSCs. Briefly, this is done by dissociating the cells and then using flow cytometry to isolate the cancer stem cells as well as the other non-tumorigenic cancer cells. Markers of CSC are known, as referenced herein. For example, colon cancer CSC may be identified as ESA+CD44+CD166+Lineage−. The ROS levels are measured in both populations using the fluorescent dye DCF-DA.

In an alternative embodiment, a gene expression data-set that is predictive is determined. Normal array technology typically requires thousand cells, but we will likely only be able to obtain a few hundred cancer stem cells from a biopsy specimen. A digital assay is used to measure the relative expression of ROS pathway genes. This gene set consists of −10 genes. Since we can easily assay 48 genes using this technique, several other genes will be incorporated in this gene set. This will include other genes that have been associated with radiation resistance including: anti-apoptotic genes (such as BCL-2, BCL-X and IAPs) and pro-apoptotic genes (such as BAX, PUMA and SMAC/DIABLO), DNA repair/radiation sensitivity genes such as ATM, CHK1 and CHK2. We will analyze the expression of each of these genes in 20 individual CoCSCs and 20 non-tumorigenic cancer cells from each patient.

What is claimed is:

1. A method of screening a cancer for susceptibility to cytotherapy, the method comprising:
quantitating the level of reactive oxygen species (ROS) markers in cancer stem cells (CSC) present in the cancer.

2. The method of claim 1 wherein the caner is a carcinoma.

3. The method of claim 2 wherein the carcinoma is breast carcinoma, colon carcinoma or squamous cell carcinoma.

4. The method of claim 1, wherein the ROS marker is direct quantitation of ROS species, and wherein CSC that are ROS^{low} relative to a non-tumorigenic cells have increased resistance to cytotherapy.

5. The method of claim 2, wherein the direct quantitation comprises contacting cancer stem cells with an ROS reactive dye, and monitoring the resulting dye change.

6. The method of claim 4, wherein the direct quantitation is performed in vivo.

7. The method of claim 4, wherein the direct quantitation is performed on an ex vivo tumor sample.

8. The method of claim 4 wherein the cancer stem cells are stained for one or more cell surface markers that identify cancer stem cells.

9. The method of claim 8, wherein the cancer stem cells are separated from non-tumorigenic cells by flow cytometry.

10. The method of claim 1, wherein the ROS marker is an ROS-related polynucleotide or polypeptide, and wherein CSC that have increased expression relative to a non-tumorigenic cells have increased resistance to cytotherapy.

11. The method of claim 10, wherein the ROS marker is one or more of cytochrome b alpha subunit (CYBA); prion protein (PRNP); peptide methionine sulfoxide reductase (MSRA); glutathione peroxidase 1 (GPX1); thioredoxin-interacting protein (TXNIP); superoxide dismutase 2 (SOD2); catalase (CAT); xpa gene (XPA); isocitrate dehydrogenase 1 (IDH1); and glutathione peroxidase 4 (GPX4).

12. The method of claim 11, wherein the direct quantitation is performed on an ex vivo tumor sample.

13. The method of claim 11 wherein the cancer stem cells are stained for one or more cell surface markers that identify cancer stem cells.

14. The method of claim 13, wherein the cancer stem cells are separated from non-tumorigenic cells by flow cytometry.

15. A method of screening a candidate chemotherapeutic agent for effectiveness against an CSC, the method comprising:
contacting said agent with the CSC, and
determining the effectiveness of said agent in increasing intracellular levels of reactive oxygen species.
